(19) United States
(12) Patent Application Publication
(10) Pub. No.: US 2026/0188471 A1
(43) Pub. Date: Jul. 2, 2026

(54) Blood Bank Turnaround Time Monitor

(71) Applicant: Oracle International Corporation, Redwood Shores, CA (US)

(72) Inventor: Mahesh Chamalapur HONNAGIRIGOWDA, Melbourne (AU)

(21) Appl. No.: 19/314,248

(22) Filed: Aug. 29, 2025

Related U.S. Application Data
(60) Provisional application No. 63/739,926, filed on Dec. 30, 2024.

(51) Int. Cl. G16H 40/20; G16H 10/40; G16H 10/60; G16H 40/67

(52) U.S. Cl. CPC G16H 40/20; G16H 10/40; G16H 10/60; G16H 40/67

(57) ABSTRACT

Embodiments operate a blood bank. Embodiments receive a request for a blood product corresponding to a patient and determine whether the request is a routine request or an emergency request. Embodiments determine an availability of patient history for the patient and display a color coded listing of parameters corresponding to the request, the parameters including a different color for blood product requests when a designated time has elapsed.

### CROSS REFERENCE TO RELATED APPLICATIONS

This application claims priority to U.S. Provisional Patent Application Ser. No. 63/739,926, filed on Dec. 30, 2024, the disclosure of which is hereby incorporated by reference.

### FIELD

One embodiment is directed generally to a blood bank monitoring system, and in particular to a blood bank turnaround time monitor.

### BACKGROUND INFORMATION

A blood bank turnaround time (“TAT”) monitor is a tool or system used in clinical laboratories and hospital blood banks to track, evaluate, and improve the efficiency and speed of processes related to the preparation and delivery of blood products. It ensures timely availability of blood products while maintaining safety and compliance with medical and regulatory standards.

Key tracking components of a blood bank TAT monitor includes an order receipt of the time when a request for blood or blood products is received, processing and preparation to track the time taken for typing, crossmatching, and preparing the required blood product, and the delivery time from preparation to the blood product being delivered to the requesting department or patient.

### SUMMARY

Embodiments operate a blood bank. Embodiments receive a request for a blood product corresponding to a patient and determine whether the request is a routine request or an emergency request. Embodiments determine an availability of patient history for product crossmatching and display a color coded listing of parameters corresponding to the request, the parameters including a different color for blood product requests when a designated time has elapsed.

### BRIEF DESCRIPTION OF THE DRAWINGS

The accompanying drawings, which are incorporated in and constitute a part of the specification, illustrate various systems, methods, and other embodiments of the disclosure. It will be appreciated that the illustrated element boundaries (e.g., boxes, groups of boxes, or other shapes) in the figures represent one embodiment of the boundaries. In some embodiments one element may be designed as multiple elements or that multiple elements may be designed as one element. In some embodiments, an element shown as an internal component of another element may be implemented as an external component and vice versa. Further, elements may not be drawn to scale.

FIG. 1 illustrates an example of a cloud based system that includes a blood bank TAT monitor system in accordance to embodiments.

FIG. 2 is a block diagram of the blood bank TAT monitor system of FIG. 1 in the form of a computer server/system in accordance to an embodiment of the present invention.

FIGS. 3 and 4 are a flow diagram of the functionality of the blood bank TAT monitor system of FIG. 1 when providing blood bank TAT monitoring in accordance to embodiments.

FIG. 5 illustrates the display of details on a user interface in accordance to embodiments.

FIG. 6 illustrates an example of how the patient product compatibility functions for Red Cells in accordance with embodiments.

FIGS. 7-10 illustrate an example cloud infrastructure that can implement the blood bank TAT monitor system of FIG. 1 in accordance to embodiments.

### DETAILED DESCRIPTION

One embodiment is a blood bank turnaround time (“TAT”) monitor that provides hospital blood management by providing an extensive overview of all blood product requests from various patient locations throughout the facility. By consolidating data from diverse inventories, embodiments produce a real-time snapshot of blood demand, allowing healthcare professionals to efficiently track requests and respond with urgency, particularly in life-threatening scenarios.

Reference will now be made in detail to the embodiments of the present disclosure, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, and circuits have not been described in detail so as not to unnecessarily obscure aspects of the embodiments. Wherever possible, like reference numbers will be used for like elements.

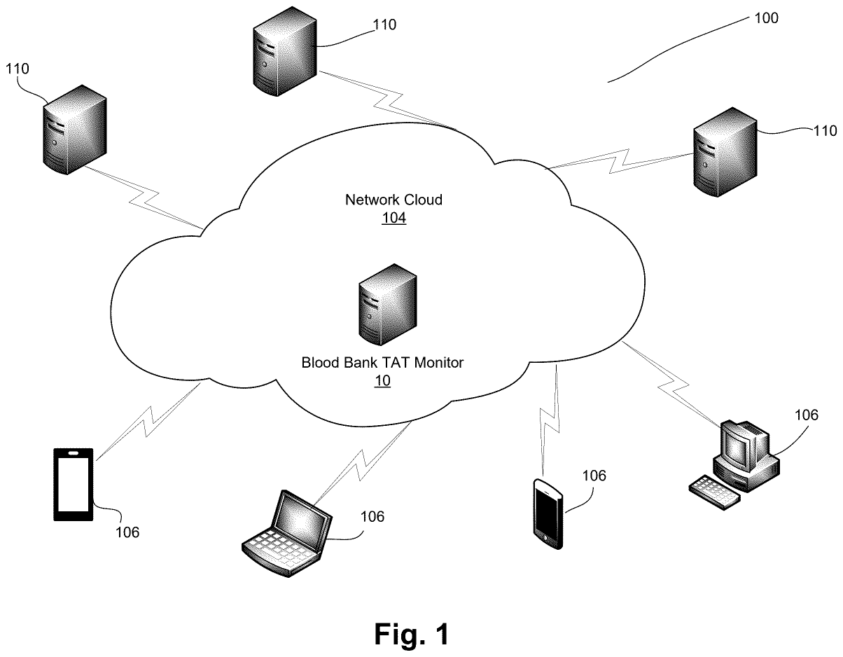

FIG. 1 illustrates an example of a system 100 that includes a blood bank TAT monitor system 10 in accordance to embodiments. Blood bank TAT monitor system 10 may be implemented within a computing environment that includes a communication network/cloud 104. Network 104 may be a private network that can communicate with a public network (e.g., the Internet) to access additional services 110 provided by a cloud services provider. Examples of communication networks include a mobile network, a wireless network, a cellular network, a local area network (“LAN”), a wide area network (“WAN”), other wireless communication networks, or combinations of these and other networks. Blood bank TAT monitor system 10 may be administered by a service provider, such as via the Oracle Cloud Infrastructure (“OCI”) from Oracle Corp.

Tenants of the cloud services provider can be companies or any type of organization or groups whose members include users of services offered by the service provider. Services may include or be provided as access to, without limitation, an application, a resource, a file, a document, data, media, or combinations thereof. Users may have individual accounts with the service provider and organizations may have enterprise accounts with the service provider, where an enterprise account encompasses or aggregates a number of individual user accounts.

System 100 further includes client devices 106, which can be any type of device that can access network 104 and can obtain the benefits of the functionality of blood bank TAT monitor system 10 of providing blood bank TAT monitoring. As disclosed herein, a “client” (also disclosed as a “client system” or a “client device”) may be a device or an application executing on a device. System 100 includes a number of different types of client devices 106 that each is able to communicate with network 104.

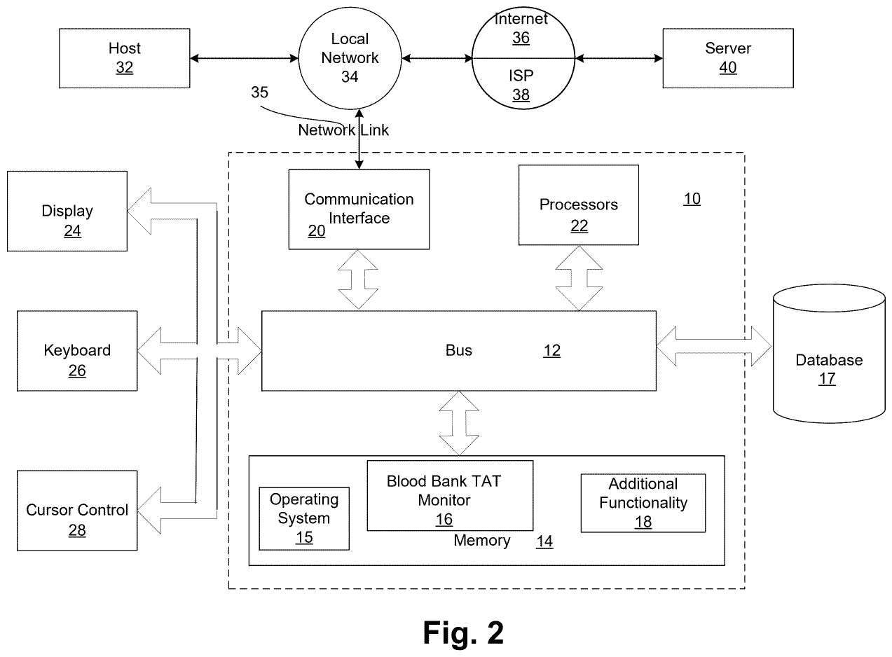

FIG. 2 is a block diagram of blood bank TAT monitor system 10 of FIG. 1 in the form of a computer server/system 10 in accordance to an embodiment of the present invention. Although shown as a single system, the functionality of system 10 can be implemented as a distributed system. Further, the functionality disclosed herein can be implemented on separate servers or devices that may be coupled together over a network. Further, one or more components of system 10 may not be included. One or more components of FIG. 2 can also be used to implement any of the elements of FIG. 1.

System 10 includes a bus 12 or other communication mechanism for communicating information, and a processor 22 coupled to bus 12 for processing information. Processor 22 may be any type of general or specific purpose processor. System 10 further includes a memory 14 for storing information and instructions to be executed by processor 22. Memory 14 can be comprised of any combination of random access memory (“RAM”), read only memory (“ROM”), static storage such as a magnetic or optical disk, or any other type of computer readable media. System 10 further includes a communication interface 20, such as a network interface card, to provide access to a network. Therefore, a user may interface with system 10 directly, or remotely through a network, or any other method.

Computer readable media may be any available media that can be accessed by processor 22 and includes both volatile and nonvolatile media, removable and non-removable media, and communication media. Communication media may include computer readable instructions, data structures, program modules, or other data in a modulated data signal such as a carrier wave or other transport mechanism, and includes any information delivery media.

Processor 22 is further coupled via bus 12 to a display 24, such as a Liquid Crystal Display (“LCD”). A keyboard 26 and a cursor control device 28, such as a computer mouse, are further coupled to bus 12 to enable a user to interface with system 10.

In one embodiment, memory 14 stores software modules that provide functionality when executed by processor 22. The modules include an operating system 15 that provides operating system functionality for system 10. The modules further include a blood bank TAT monitor module 16 that provides blood bank TAT monitoring, and all other functionality disclosed herein. System 10 can be part of a larger system. Therefore, system 10 can include one or more additional functional modules 18, such as an electronic medical records (“EMR”) integrated solution. A file storage device or database 17 is coupled to bus 12 to provide centralized storage for modules 16 and 18, including patient data, historical procedures, physician records, etc. In one embodiment, database 17 is a relational database management system (“RDBMS”) that can use Structured Query Language (“SQL”) to manage the stored data.

In embodiments, communication interface 20 provides a two-way data communication coupling to a network link 35 that is connected to a local network 34. For example, communication interface 20 may be an integrated services digital network (“ISDN”) card, cable modem, satellite modem, or a modem to provide a data communication connection to a corresponding type of telephone line or Ethernet. As another example, communication interface 20 may be a local area network (“LAN”) card to provide a data communication connection to a compatible LAN. Wireless links may also be implemented. In any such implementation, communication interface 20 sends and receives electrical, electromagnetic or optical signals that carry digital data streams representing various types of information.

Network link 35 typically provides data communication through one or more networks to other data devices. For example, network link 35 may provide a connection through local network 34 to a host computer 32 or to data equipment operated by an Internet Service Provider (“ISP”) 38. ISP 38 in turn provides data communication services through the Internet 36. Local network 34 and Internet 36 both use electrical, electromagnetic or optical signals that carry digital data streams. The signals through the various networks and the signals on network link 35 and through communication interface 20, which carry the digital data to and from computer system 10, are example forms of transmission media.

System 10 can send messages and receive data, including program code, through the network(s), network link 35 and communication interface 20. In the Internet example, a server 40 might transmit a requested code for an application program through Internet 36, ISP 38, local network 34 and communication interface 20. The received code may be executed by processor 22 as it is received, and/or stored in database 17, or other non-volatile storage for later execution.

In one embodiment, system 10 is a computing/data processing system including an application or collection of distributed applications for enterprise organizations, and may also implement logistics, manufacturing, and inventory management functionality. The applications and computing system 10 may be configured to operate locally or be implemented as a cloud-based networking system, for example in an infrastructure-as-a-service (“IAAS”), platform-as-a-service (“PAAS”), software-as-a-service (“SAAS”) architecture, or other type of computing solution.

As disclosed, a blood bank TAT monitor is a tool or system used in clinical laboratories and hospital blood banks to track, evaluate, and improve the efficiency and speed of processes related to the preparation and delivery of blood products. The lack of a TAT monitor in a blood bank, especially regarding requests for blood products from clinicians, can severely disrupt the efficiency of blood procurement and dispensing activities. This shortcoming results in significant delays in obtaining the appropriate blood products and negatively impacts the overall turnaround times for blood bank testing and product distribution.

In the absence of an effective monitoring system, blood bank staff often lack the necessary tools to track and manage the timelines for requests and deliveries, leading to poor communication and coordination with clinical teams. Additionally, this inefficiency places extra pressure on blood bank resources, as staff may struggle to meet requests quickly, which exacerbates the delays. The consequences of these delays extend beyond individual patient care, potentially compromising the quality of healthcare delivery and straining the relationships between clinical teams and the blood bank.

Embodiments provide the inventory location of compatible product by enabling the blood bank to rapidly initiate procurement from other facilities within a network if necessary and facilitate the monitoring and management of blood products availability across multiple hospitals to enhance patient care. Embodiments examine the patient's historical and current laboratory results, search for compatible products within the existing inventory, and access other inventories across multiple hospitals within the same network, subsequently recommending suitable products for the patient.

Embodiments recommend suitable products for cross-matching (“XM”) based on historical data by analyzing current inventory alongside historical patient data, therefore enabling blood banks to swiftly and precisely pinpoint the required blood units and reducing the time needed for crossmatching. In embodiments, crossmatching is implemented per AABB guidelines disclosed below. In other embodiments, a trained machine learning model is used for generating recommendations.

Embodiments provide alerts for emergency/mass transfusion protocol. Critical scenarios such as a massive transfusion protocol (“MTP”, e.g., a rapid administration of large amounts of blood products (at least 6 units of PRBC) in fixed ratios (usually 1:1:1) for the management of hemorrhagic shock) and similar emergencies require both accuracy and speed. Embodiments provide timely alerts that assist in administering the blood units in the stipulated time frames. By implementing specific alerts for different types of product requests—where routine requests do not trigger alerts but emergency and MTP alerts do—scientists or other blood bank personnel can respond quickly by prioritizing these urgent requests over less pressing tasks.

Embodiments display special requirements/prompts, such as clinical diagnosis details and other product request information provided by the ordering physician (e.g., if physician requests bag of red blood cells, embodiments include the reason for the request, previous transfusions, etc.).

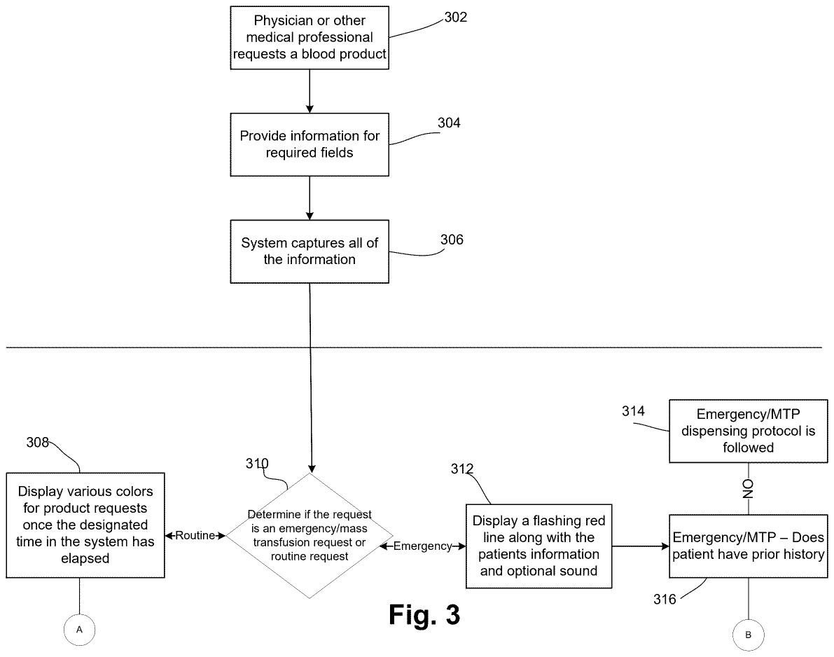

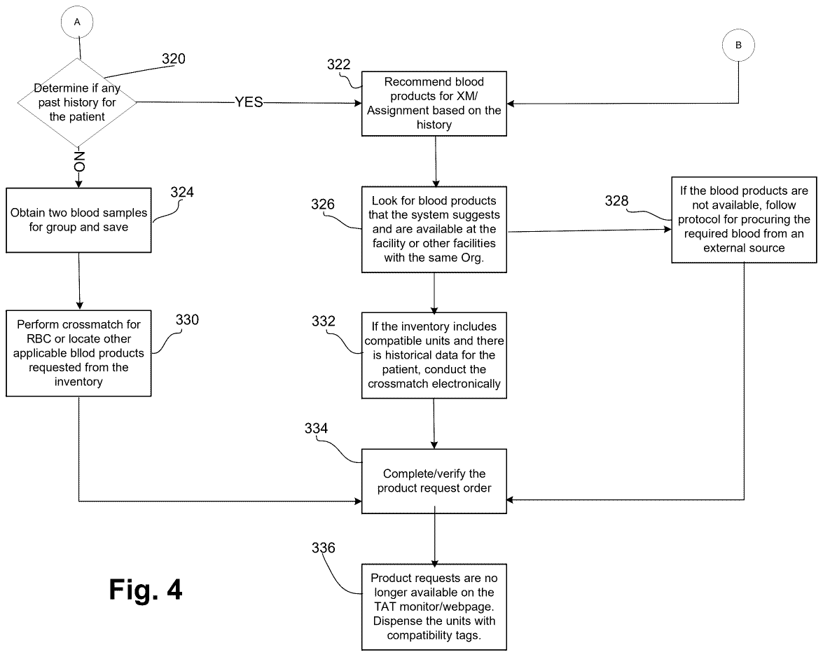

FIGS. 3 and 4 are a flow diagram of the functionality of blood bank TAT monitor system 10 of FIG. 1 when providing blood bank TAT monitoring in accordance to embodiments. In one embodiment, the functionality of the flow/block diagram of FIGS. 3 and 4 is implemented by software stored in memory or other computer readable or tangible medium, and executed by a processor. In other embodiments, the functionality may be performed by hardware (e.g., through the use of an application specific integrated circuit (“ASIC”), a programmable gate array (“PGA”), a field programmable gate array (“FPGA”), etc.), or any combination of hardware and software.

At 302, a physician or other medical professional orders a blood product (e.g., red blood cells, plasma, platelets, etc.).

At 304, information for required fields are provided (e.g., details justifying the request, history of transfusions, etc.).

At 306, system 10 captures all of the information.

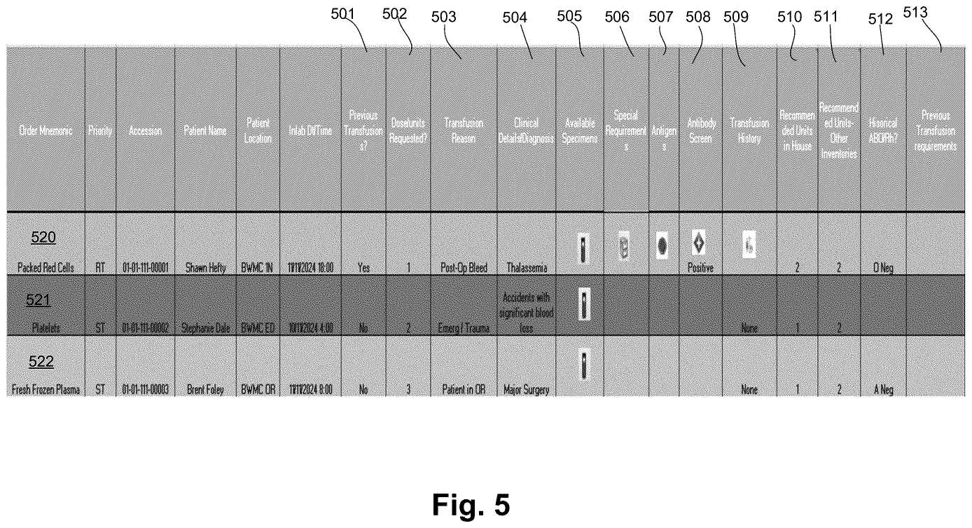

At 310, it is determined if the request is an emergency/mass transfusion request or routine request. If routine at 310, at 308, system 10 displays various colors for product requests once the designated time in the system has elapsed. For example, for any blood bank product requests made by physicians or other personnel, embodiments predefine time limits to be set for each order. If an order is not addressed by the blood bank within these time frames, it will be highlighted in different colors. For example, a routine order that is not processed within the first 15 minutes will appear with a grey background. If the order remains unaddressed after 30 minutes, it will change to yellow/orange (e.g., row 522 of FIG. 5), and if it is not processed and results are not available after one hour, it will be displayed in red (e.g., row 521 of FIG. 5).

At 320, it is checked if there are any historical results for the patient (e.g., previous Blood Group(s), Antibody Screen, etc.). If yes at 322, blood products are recommended based on the history. If no history, at 324 two blood samples (or any other predefined number) are obtained. At 330, a crossmatch is conducted for red blood cells (“RBC”) or other applicable blood products are located and requested from inventory.

At 326, system 10 looks for suggest blood product at the facilities (i.e., as part of inventory) or other facilities at the same organization. At 328, if the necessary blood products are not available, protocol is followed for procuring the products from an external source. At 332, if the inventory includes compatible units and there is historical data for the patient in the system that satisfies all the criteria set for a computer crossmatch (disclosed below), lab scientists will review the suggested units and conduct the crossmatch electronically. At 334, the blood bank completes the blood product request order(s), and the nurses are notified to collect the blood bags. The units are dispensed with compatibility tags once the nurses arrive at the blood bank. At 336, the product requests are no longer visible on the TAT monitor.

If at 310, if an emergency or MTP, at 312 a flashing red line is displayed, along with the patients information and optional sound. At 316, it is determined if prior history for the patient exists. If no, at 314, emergency/MTP dispensing protocol is followed. If history, functionality resumes at 322.

At the completion of the functionality of FIGS. 3 and 4 (or during the functionality), system 10 displays all relevant details with color coding or some other distinguishing markings. FIG. 5 illustrates the display of details on a user interface in accordance to embodiments. Novel fields (i.e., fields not generated and displayed by known blood bank monitor systems) that are generated, displayed and shown in FIG. 5 include Previous Transfusions? 501, Dose/Units Requested? 502, Transfusion Reasons 503, Clinical Details/Diagnosis 504, Available Specimen (valid specimen) 505, Special Requirements 506, Antigens 507, Antibody Screen results 508, Transfusion History 509, Recommended units in House 510, Recommended units in other inventories 511 and Historical ABO/Rh 512 and Previous Transfusion requirements, if any 513. Other embodiments can include customized additional fields at the patient, order, or encounter level. For the Recommended units in House 510 and Recommended units in other inventories 511, in one embodiment, when Cursor is hovered over the number of units available, a tool tip displays the unit(s) details with the Unit ABO/Rh and expiration date and time.

In the example of FIG. 5, row 520 (“Packed Red Cells”) is green, row 521 (“Platelets”) is red, and row 522 (“Fresh Frozen Plasma”) is yellow. However, any other coloring or marking scheme can be used.

In connection with 322 of FIG. 4, patient product compatibility is established within system 10 to identify the blood products that are acceptable for specific patients. The criteria will be used to ascertain and recommend the appropriate blood products. In the case of red blood cells, additional system checks will be performed for a computer crossmatch criteria (disclosed below) prior to suggesting a unit of red blood cells for crossmatching. The parameters for product-patient compatibility specify the patient ABO groups and Rh types eligible to receive a product of a particular ABO blood group and Rh blood type. The laboratory has the option to select the compatible patient ABO groups and Rh types with or without a warning. If a selection is made with a warning, laboratory users will receive an alert generated by system 10 indicating that the product corresponds to an unmatched group and type, but they may proceed with the crossmatch or dispense it if they possess the appropriate access. The laboratory can also prevent a product with a specific ABO group and Rh type from being crossmatched, assigned, or dispensed to a patient with a corresponding ABO group and Rh type by leaving it deselected.

The laboratory establishes these parameters for each product and ABO/Rh combination that can be dispensed, assigned, or crossmatched. In addition to indicating the patient ABO/Rh types that can be associated with this product—with or without a warning—it is necessary to specify whether the product can be crossmatched with or dispensed to a patient with unknown (blank) ABO/Rh status (options include Yes, No, or Yes With Warning).

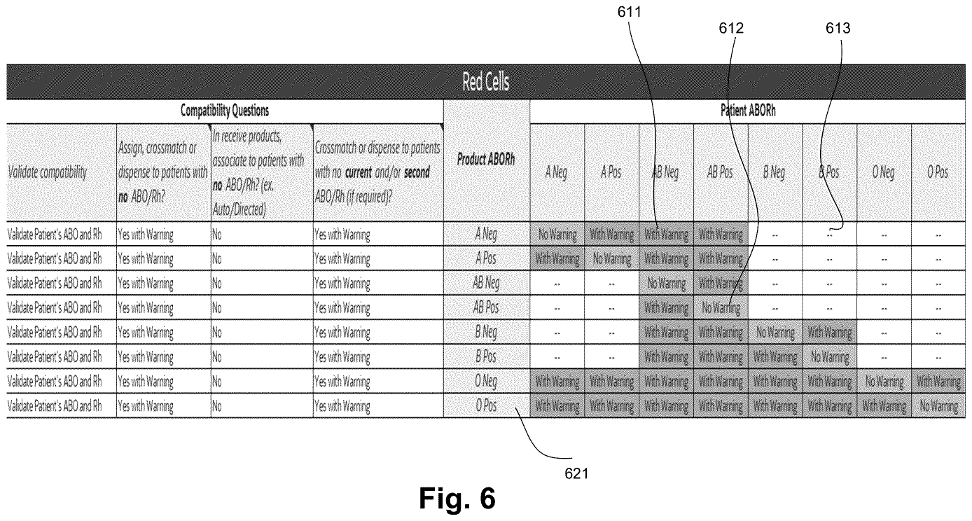

By using the pattern set up by the clients in system 10, recommendations for suitable products from the inventory for crossmatching will be generated by embodiments. FIG. 6 illustrates an example of how the patient product compatibility functions for Red Cells in accordance with embodiments.

The product-patient compatibility parameters shown in FIG. 6 define the patient ABO groups and Rh types that can receive a product with a specific ABO group and Rh type. The compatible patient ABO groups and Rh types can be selected with (e.g., 611) or without (e.g., 612) a warning. If selected with a warning, a warning is given that the product is of an unmatched group and type, but the crossmatch is allowed to continue or dispense if the user has the proper level of security. The user can prevent a product with a specific ABO group and Rh type from being crossmatched, assigned, or dispensed to a specific patient ABO group and Rh type by leaving it deselected (e.g., 613).

Clients/users can provide these parameters to system 10 for every product and ABO/Rh combination that can be dispensed, assigned, or crossmatched. In addition to listing at col. 621 the patient ABO/Rh types that can be associated with this product with or without a warning, the user indicates whether the user can crossmatch the product with or dispense the product to a patient with an unknown (blank) ABO/Rh (e.g., select Yes, No, or Yes With Warning).

Embodiments compare the Product ABORh column 621 is compared to the Patient ABORh row:
(a) Can an A Neg red cell product be crossmatched with an A Neg patient?—The response is ‘No Warning,’ indicating system 10 permits the crossmatch since the product and patient blood groups match.
(b) In a case where the product is A Pos and the patient is A Neg, system 10 is configured to issue a warning during the crossmatch—Response is ‘With Warning.’
(c) Similarly, A Neg patients cannot receive AB Neg red cells, resulting in a hard stop for users in the system—Response is ‘—’ indicating a hard stop.

The computer crossmatch implemented by embodiments at 332 of FIG. 4 is a viable option for patients who fulfill specific criteria. These criteria are established within system 10 based on its preferences, and patients who do not satisfy these criteria will be ineligible for a computer crossmatch. Embodiments will conduct the necessary evaluations to determine each patient's eligibility, and those who do not qualify for this functionality cannot be overridden by end users. To utilize the computer crossmatch functionality, an up-to-date ABO/Rh sample and a second determinant in the record are required in embodiments. It is advisable to configure the system settings to mandate testing of the second determinant using a current sample or by comparing it with an outdated ABO/Rh record. Retesting the same sample for the second determinant is not recommended. For a more stringent workflow, embodiments also suggest disallowing overrides in cases where there are discrepancies between the initial ABO/Rh determination and the patient's demographic ABO/Rh, inconsistencies between the two ABO/Rh determinants, if the patient has a current positive antibody screen, or if there is a clinically significant antibody documented in the patient's record. In general, the computer crossmatch feature offers users a rapid and effective way to crossmatch blood products. Utilizing preference questions, this functionality ensures both flexibility and security at a granular level, all while conducting swift and precise eligibility checks.

Recommendations in embodiments are in accordance with the guidelines set by the U.S. Food and Drug Administration (“FDA”), Association for the Advancement of Blood & Biotherapies (“AABB”), and various other regulatory bodies. Below are guidelines from both the FDA and AABB that outline the standards for computer crossmatching and are implemented in embodiments, such to changes in response to changes in standards:

### FDA Standards on Computer Crossmatch:

The recipient ABO/Rh (D) Type and Interpretation You should determine a recipient's ABO and Rh (D) antigens (Ref. 11). You should either perform or maintain a record of a second test, confirming the recipient's ABO/Rh (D). For example, this second test may be a record of a test performed previously, or a repeat test on a second, separately drawn specimen. Repeating ABO and Rh (D) tests on the same specimen is not recommended, as the major cause of ABO errors is “wrong blood in tube” (WBIT). Performing tests on two separately drawn specimens is preferred, as this lessens the likelihood of errors because specimens have been drawn in error. In certain situations, however, only one specimen may be available for testing, such as in emergencies or when only one sample is received for home transfusion. At those times, repeat testing may be performed on the same specimen, but the repeat test should be performed either by a different technologist or by the same technologist using different reagents. If ABO typing discrepancies exist, you should not rely on a computer crossmatch. This is particularly important if there is mixed field red cell reactivity, missing serum reactivity, or apparent change in blood type following hematopoietic stem cell transplantation. Under those circumstances, your procedures should provide for compatibility testing using serologic crossmatch techniques.

### AABB Standards on Computer Crossmatch:

5.15.2 Computer Crossmatch—If a computer system is used to detect ABO incompatibility, the following requirements shall be met:
   5.15.2.1 The computer system has been validated on site to ensure that only ABO-compatible Whole Blood or Red Blood Cell components have been selected for transfusion.
   5.15.2.2 Two determinations of the recipient's ABO group as specified in Standard 5.13.1 are made, one on a current sample and the second by one of the following methods: by retesting the same sample, by testing a second current sample, or by comparison with previous records. Standard 5.11 applies.
   5.15.2.3 The system contains the donation identification number, component name, ABO group, and Rh type of the component; the confirmed unit ABO group; the two unique recipient identifiers; recipient ABO group, Rh type, and antibody screen results; and interpretation of compatibility.
   5.15.2.4 A method exists to verify correct entry of data before release of blood or components.
   5.15.2.5 The system contains logic to alert the user to discrepancies between the donor ABO group and Rh type on the unit label and those determined by blood group confirmatory tests and to ABO incompatibility between the recipient and the donor unit.

Embodiments implement a Turn Around Time monitoring system for blood product requests, which allows for more efficient oversight and quicker response capabilities. Embodiments can significantly enhance overall productivity and improve patient care and safety, especially during critical medical procedures. By establishing a thorough turnaround time tracking system, healthcare providers can meticulously oversee the entire process of blood products, from collection and testing to transfusion and post-transfusion support, ensuring every unit of blood is meticulously accounted for and utilized effectively. This real-time monitoring enables medical staff to swiftly identify any delays or challenges that may occur, such as blood type compatibility issues or the timely availability of necessary blood products, allowing for prompt and informed interventions.

These proactive measures not only help minimize the risk of adverse events but also streamline operational workflows by fostering better collaboration among various departments, including laboratories, blood banks, and clinical units. By encouraging a culture of efficiency and accountability, healthcare organizations can significantly reduce waiting times, ensuring that patients receive vital blood transfusions when needed, which can prove to be life-saving. Consequently, the synergy of effective monitoring and timely response contributes to a more robust blood management system that addresses patients'urgent needs while prioritizing their safety and well-being, ultimately boosting confidence in the healthcare system as a whole. This comprehensive approach sets a new standard for blood management practices, highlighting the importance of timely interventions in delivering high-quality care during the most critical stages of patient treatment.

Known solutions enable users to track order priority and departmental status. In contrast, embodiments can transform the approach to managing blood bank orders and inventory. Known solutions fail to utilize essential clinician-documented information at the time of the request and do not incorporate previous data, such as Blood Group and Antibody Screen results. Embodiments allow for streamline order processing and improve clinical decision-making by proposing relevant blood products from the inventory for cross-matching based on the patient's urgent needs.

The use of embodiments for blood bank product requests marks a major improvement in blood product management, especially during critical situations such as MTP or emergencies where accuracy and speed are essential. In these high-pressure environments, the ability to obtain blood products quickly can significantly influence patient outcomes. By comparing current inventory with historical patient data, the system allows blood banks to quickly and accurately identify the necessary blood units, thereby minimizing the time required for crossmatching. If the required blood units are not in stock or have specific conditions, embodiments enable blood banks to swiftly initiate procurement from external suppliers or other facilities within the network. This capability to track and manage blood product availability across multiple hospitals promotes collaboration and resource sharing greatly enhances patient care. When hospitals within the same organization utilize this monitoring system, it delivers real-time information on inventory levels, encouraging a unified approach to addressing urgent blood needs. The removal of geographical limitations facilitates broader searches and sharing of compatible units, particularly in complex scenarios with special requirements. Ultimately, The blood bank turnaround time monitor in accordance to embodiments not only simplifies blood product requests but also fosters a culture of effective resource management that can save lives in critical situations and improve overall healthcare outcomes.

### Example Cloud Infrastructure

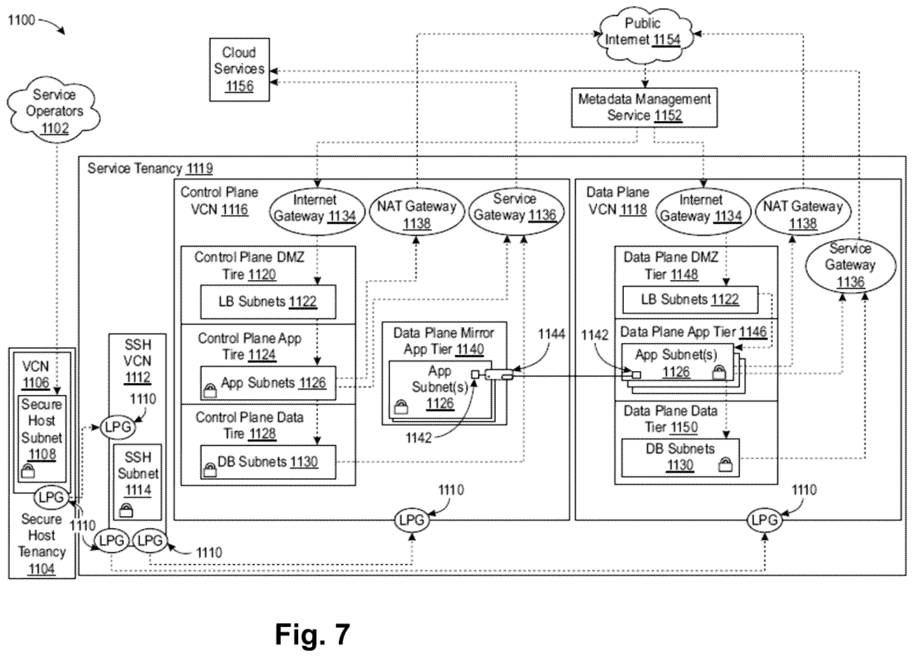

FIGS. 7-10 illustrate an example cloud infrastructure that can implement system 100 that can include blood bank TAT monitor system 10 of FIG. 1 in accordance to embodiments. The use of the cloud infrastructure, as opposed to an on-premise implementation, allows for blood bank inventory data, and other data, to be receive from many different users and sources that are interacting with system 10.

As disclosed above, infrastructure as a service (“IaaS”) is one particular type of cloud computing. IaaS can be configured to provide virtualized computing resources over a public network (e.g., the Internet). In an IaaS model, a cloud computing provider can host the infrastructure components (e.g., servers, storage devices, network nodes (e.g., hardware), deployment software, platform virtualization (e.g., a hypervisor layer), or the like). In some cases, an IaaS provider may also supply a variety of services to accompany those infrastructure components (e.g., billing, monitoring, logging, security, load balancing and clustering, etc.). Thus, as these services may be policy-driven, IaaS users may be able to implement policies to drive load balancing to maintain application availability and performance.

In some instances, IaaS customers may access resources and services through a wide area network (“WAN”), such as the Internet, and can use the cloud provider's services to install the remaining elements of an application stack. For example, the user can log in to the IaaS platform to create virtual machines (“VM”), install operating systems (“OS” ) on each VM, deploy middleware such as databases, create storage buckets for workloads and backups, and even install enterprise software into that VM. Customers can then use the provider's services to perform various functions, including balancing network traffic, troubleshooting application issues, monitoring performance, managing disaster recovery, etc.

In most cases, a cloud computing model will require the participation of a cloud provider. The cloud provider may, but need not be, a third-party service that specializes in providing (e.g., offering, renting, selling) IaaS. An entity might also opt to deploy a private cloud, becoming its own provider of infrastructure services.

In some examples, IaaS deployment is the process of putting a new application, or a new version of an application, onto a prepared application server or the like. It may also include the process of preparing the server (e.g., installing libraries, daemons, etc.). This is often managed by the cloud provider, below the hypervisor layer (e.g., the servers, storage, network hardware, and virtualization). Thus, the customer may be responsible for handling (OS), middleware, and/or application deployment (e.g., on self-service virtual machines (e.g., that can be spun up on demand)) or the like.

In some examples, IaaS provisioning may refer to acquiring computers or virtual hosts for use, and even installing needed libraries or services on them. In most cases, deployment does not include provisioning, and the provisioning may need to be performed first.

In some cases, there are two different problems for IaaS provisioning. First, there is the initial challenge of provisioning the initial set of infrastructure before anything is running. Second, there is the challenge of evolving the existing infrastructure (e.g., adding new services, changing services, removing services, etc.) once everything has been provisioned. In some cases, these two challenges may be addressed by enabling the configuration of the infrastructure to be defined declaratively. In other words, the infrastructure (e.g., what components are needed and how they interact) can be defined by one or more configuration files. Thus, the overall topology of the infrastructure (e.g., what resources depend on which, and how they each work together) can be described declaratively. In some instances, once the topology is defined, a workflow can be generated that creates and/or manages the different components described in the configuration files.

In some examples, an infrastructure may have many interconnected elements. For example, there may be one or more virtual private clouds (“VPC”) (e.g., a potentially on-demand pool of configurable and/or shared computing resources), also known as a core network. In some examples, there may also be one or more security group rules provisioned to define how the security of the network will be set up and one or more virtual machines. Other infrastructure elements may also be provisioned, such as a load balancer, a database, or the like. As more and more infrastructure elements are desired and/or added, the infrastructure may incrementally evolve.

In some instances, continuous deployment techniques may be employed to enable deployment of infrastructure code across various virtual computing environments. Additionally, the described techniques can enable infrastructure management within these environments. In some examples, service teams can write code that is desired to be deployed to one or more, but often many, different production environments (e.g., across various different geographic locations, sometimes spanning the entire world). However, in some examples, the infrastructure on which the code will be deployed must first be set up. In some instances, the provisioning can be done manually, a provisioning tool may be utilized to provision the resources, and/or deployment tools may be utilized to deploy the code once the infrastructure is provisioned.

FIG. 7 is a block diagram 1100 illustrating an example pattern of an IaaS architecture, according to at least one embodiment. Service operators 1102 can be communicatively coupled to a secure host tenancy 1104 that can include a virtual cloud network (“VCN”) 1106 and a secure host subnet 1108. In some examples, the service operators 1102 may be using one or more client computing devices, which may be portable handheld devices (e.g., an iPhone®, cellular telephone, an iPad®, computing tablet, a personal digital assistant (“PDA”)) or wearable devices (e.g., a Meta Quest® head mounted display), running software such as Microsoft Windows Mobile®, and/or a variety of mobile operating systems such as iOS, Windows Phone, Android, BlackBerry 8, Palm OS, and the like, and being Internet, e-mail, short message service (“SMS”), Blackberry®, or other communication protocol enabled. Alternatively, the client computing devices can be general purpose personal computers including, by way of example, personal computers and/or laptop computers running various versions of Microsoft Windows®, Apple Macintosh®, and/or Linux operating systems. The client computing devices can be workstation computers running any of a variety of commercially-available UNIX® or UNIX-like operating systems, including without limitation the variety of GNU/Linux operating systems, such as for example, Google Chrome OS. Alternatively, or in addition, client computing devices may be any other electronic device, such as a thin-client computer, an Internet-enabled gaming system (e.g., a Microsoft Xbox gaming console with or without a Kinect® gesture input device), and/or a personal messaging device, capable of communicating over a network that can access the VCN 1106 and/or the Internet.

The VCN 1106 can include a local peering gateway (“LPG”) 1110 that can be communicatively coupled to a secure shell (“SSH”) VCN 1112 via an LPG 1110 contained in the SSH VCN 1112. The SSH VCN 1112 can include an SSH subnet 1114, and the SSH VCN 1112 can be communicatively coupled to a control plane VCN 1116 via the LPG 1110 contained in the control plane VCN 1116. Also, the SSH VCN 1112 can be communicatively coupled to a data plane VCN 1118 via an LPG 1110. The control plane VCN 1116 and the data plane VCN 1118 can be contained in a service tenancy 1119 that can be owned and/or operated by the IaaS provider.

The control plane VCN 1116 can include a control plane demilitarized zone (“DMZ”) tier 1120 that acts as a perimeter network (e.g., portions of a corporate network between the corporate intranet and external networks). The DMZ-based servers may have restricted responsibilities and help keep security breaches contained. Additionally, the DMZ tier 1120 can include one or more load balancer (“LB”) subnet(s) 1122, a control plane app tier 1124 that can include app subnet(s) 1126, a control plane data tier 1128 that can include database (DB) subnet(s) 1130 (e.g., frontend DB subnet(s) and/or backend DB subnet(s)). The LB subnet(s) 1122 contained in the control plane DMZ tier 1120 can be communicatively coupled to the app subnet(s) 1126 contained in the control plane app tier 1124 and an Internet gateway 1134 that can be contained in the control plane VCN 1116, and the app subnet(s) 1126 can be communicatively coupled to the DB subnet(s) 1130 contained in the control plane data tier 1128 and a service gateway 1136 and a network address translation (NAT) gateway 1138. The control plane VCN 1116 can include the service gateway 1136 and the NAT gateway 1138.

The control plane VCN 1116 can include a data plane mirror app tier 1140 that can include app subnet(s) 1126. The app subnet(s) 1126 contained in the data plane mirror app tier 1140 can include a virtual network interface controller (VNIC) 1142 that can execute a compute instance 1144. The compute instance 1144 can communicatively couple the app subnet(s) 1126 of the data plane mirror app tier 1140 to app subnet(s) 1126 that can be contained in a data plane app tier 1146.

The data plane VCN 1118 can include the data plane app tier 1146, a data plane DMZ tier 1148, and a data plane data tier 1150. The data plane DMZ tier 1148 can include LB subnet(s) 1122 that can be communicatively coupled to the app subnet(s) 1126 of the data plane app tier 1146 and the Internet gateway 1134 of the data plane VCN 1118. The app subnet(s) 1126 can be communicatively coupled to the service gateway 1136 of the data plane VCN 1118 and the NAT gateway 1138 of the data plane VCN 1118. The data plane data tier 1150 can also include the DB subnet(s) 1130 that can be communicatively coupled to the app subnet(s) 1126 of the data plane app tier 1146.

The Internet gateway 1134 of the control plane VCN 1116 and of the data plane VCN 1118 can be communicatively coupled to a metadata management service 1152 that can be communicatively coupled to public Internet 1154. Public Internet 1154 can be communicatively coupled to the NAT gateway 1138 of the control plane VCN 1116 and of the data plane VCN 1118. The service gateway 1136 of the control plane VCN 1116 and of the data plane VCN 1118 can be communicatively coupled to cloud services 1156.

In some examples, the service gateway 1136 of the control plane VCN 1116 or of the data plane VCN 1118 can make application programming interface (“API”) calls to cloud services 1156 without going through public Internet 1154. The API calls to cloud services 1156 from the service gateway 1136 can be one-way: the service gateway 1136 can make API calls to cloud services 1156, and cloud services 1156 can send requested data to the service gateway 1136. But, cloud services 1156 may not initiate API calls to the service gateway 1136.

In some examples, the secure host tenancy 1104 can be directly connected to the service tenancy 1119, which may be otherwise isolated. The secure host subnet 1108 can communicate with the SSH subnet 1114 through an LPG 1110 that may enable two-way communication over an otherwise isolated system. Connecting the secure host subnet 1108 to the SSH subnet 1114 may give the secure host subnet 1108 access to other entities within the service tenancy 1119.

The control plane VCN 1116 may allow users of the service tenancy 1119 to set up or otherwise provision desired resources. Desired resources provisioned in the control plane VCN 1116 may be deployed or otherwise used in the data plane VCN 1118. In some examples, the control plane VCN 1116 can be isolated from the data plane VCN 1118, and the data plane mirror app tier 1140 of the control plane VCN 1116 can communicate with the data plane app tier 1146 of the data plane VCN 1118 via VNICs 1142 that can be contained in the data plane mirror app tier 1140 and the data plane app tier 1146.

In some examples, users of the system, or customers, can make requests, for example create, read, update, or delete (“CRUD”) operations, through public Internet 1154 that can communicate the requests to the metadata management service 1152. The metadata management service 1152 can communicate the request to the control plane VCN 1116 through the Internet gateway 1134. The request can be received by the LB subnet(s) 1122 contained in the control plane DMZ tier 1120. The LB subnet(s) 1122 may determine that the request is valid, and in response to this determination, the LB subnet(s) 1122 can transmit the request to app subnet(s) 1126 contained in the control plane app tier 1124. If the request is validated and requires a call to public Internet 1154, the call to public Internet 1154 may be transmitted to the NAT gateway 1138 that can make the call to public Internet 1154. Memory that may be desired to be stored by the request can be stored in the DB subnet(s) 1130.

In some examples, the data plane mirror app tier 1140 can facilitate direct communication between the control plane VCN 1116 and the data plane VCN 1118. For example, changes, updates, or other suitable modifications to configuration may be desired to be applied to the resources contained in the data plane VCN 1118. Via a VNIC 1142, the control plane VCN 1116 can directly communicate with, and can thereby execute the changes, updates, or other suitable modifications to configuration to, resources contained in the data plane VCN 1118.

In some embodiments, the control plane VCN 1116 and the data plane VCN 1118 can be contained in the service tenancy 1119. In this case, the user, or the customer, of the system may not own or operate either the control plane VCN 1116 or the data plane VCN 1118. Instead, the IaaS provider may own or operate the control plane VCN 1116 and the data plane VCN 1118, both of which may be contained in the service tenancy 1119. This embodiment can enable isolation of networks that may prevent users or customers from interacting with other users', or other customers', resources. Also, this embodiment may allow users or customers of the system to store databases privately without needing to rely on public Internet 1154, which may not have a desired level of security, for storage.

In other embodiments, the LB subnet(s) 1122 contained in the control plane VCN 1116 can be configured to receive a signal from the service gateway 1136. In this embodiment, the control plane VCN 1116 and the data plane VCN 1118 may be configured to be called by a customer of the IaaS provider without calling public Internet 1154. Customers of the IaaS provider may desire this embodiment since database(s) that the customers use may be controlled by the IaaS provider and may be stored on the service tenancy 1119, which may be isolated from public Internet 1154.

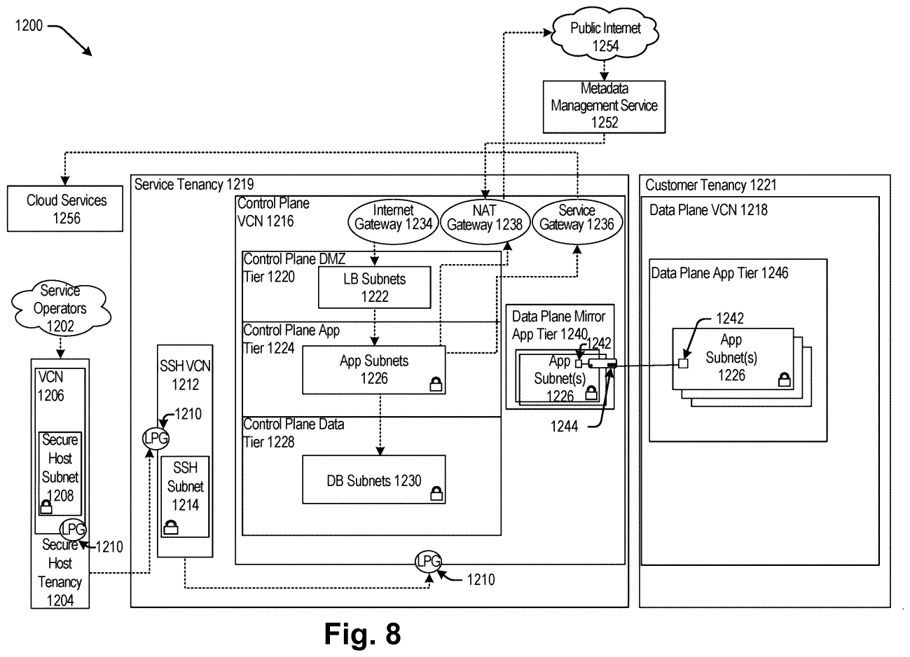

FIG. 8 is a block diagram 1200 illustrating another example pattern of an IaaS architecture, according to at least one embodiment. Service operators 1202 (e.g. service operators 1102) can be communicatively coupled to a secure host tenancy 1204 (e.g. the secure host tenancy 1104) that can include a virtual cloud network (VCN) 1206 (e.g. the VCN 1106) and a secure host subnet 1208 (e.g. the secure host subnet 1108). The VCN 1206 can include a local peering gateway (LPG) 1210 (e.g. the LPG 1110) that can be communicatively coupled to a secure shell (SSH) VCN 1212 (e.g. the SSH VCN 1112 10) via an LPG 1110 contained in the SSH VCN 1212. The SSH VCN 1212 can include an SSH subnet 1214 (e.g. the SSH subnet 1114), and the SSH VCN 1212 can be communicatively coupled to a control plane VCN 1216 (e.g. the control plane VCN 1116) via an LPG 1210 contained in the control plane VCN 1216. The control plane VCN 1216 can be contained in a service tenancy 1219 (e.g. the service tenancy 1119), and the data plane VCN 1218 (e.g. the data plane VCN 1118) can be contained in a customer tenancy 1221 that may be owned or operated by users, or customers, of the system.

The control plane VCN 1216 can include a control plane DMZ tier 1220 (e.g. the control plane DMZ tier 1120) that can include LB subnet(s) 1222 (e.g. LB subnet(s) 1122), a control plane app tier 1224 (e.g. the control plane app tier 1124) that can include app subnet(s) 1226 (e.g. app subnet(s) 1126), a control plane data tier 1228 (e.g. the control plane data tier 1128) that can include database (DB) subnet(s) 1230 (e.g. similar to DB subnet(s) 1130). The LB subnet(s) 1222 contained in the control plane DMZ tier 1220 can be communicatively coupled to the app subnet(s) 1226 contained in the control plane app tier 1224 and an Internet gateway 1234 (e.g. the Internet gateway 1134) that can be contained in the control plane VCN 1216, and the app subnet(s) 1226 can be communicatively coupled to the DB subnet(s) 1230 contained in the control plane data tier 1228 and a service gateway 1236 and a network address translation (NAT) gateway 1238 (e.g. the NAT gateway 1138). The control plane VCN 1216 can include the service gateway 1236 and the NAT gateway 1238.

The control plane VCN 1216 can include a data plane mirror app tier 1240 (e.g. the data plane mirror app tier 1140) that can include app subnet(s) 1226. The app subnet(s) 1226 contained in the data plane mirror app tier 1240 can include a virtual network interface controller (VNIC) 1242 (e.g. the VNIC of 1142) that can execute a compute instance 1244 (e.g. similar to the compute instance 1144). The compute instance 1244 can facilitate communication between the app subnet(s) 1226 of the data plane mirror app tier 1240 and the app subnet(s) 1226 that can be contained in a data plane app tier 1246 (e.g. the data plane app tier 1146) via the VNIC 1242 contained in the data plane mirror app tier 1240 and the VNIC 1242 contained in the data plane app tier 1246.

The Internet gateway 1234 contained in the control plane VCN 1216 can be communicatively coupled to a metadata management service 1252 (e.g. the metadata management service 1152) that can be communicatively coupled to public Internet 1254 (e.g. public Internet 1154). Public Internet 1254 can be communicatively coupled to the NAT gateway 1238 contained in the control plane VCN 1216. The service gateway 1236 contained in the control plane VCN 1216 can be communicatively couple to cloud services 1256 (e.g. cloud services 1156).

In some examples, the data plane VCN 1218 can be contained in the customer tenancy 1221. In this case, the IaaS provider may provide the control plane VCN 1216 for each customer, and the IaaS provider may, for each customer, set up a unique compute instance 1244 that is contained in the service tenancy 1219. Each compute instance 1244 may allow communication between the control plane VCN 1216, contained in the service tenancy 1219, and the data plane VCN 1218 that is contained in the customer tenancy 1221. The compute instance 1244 may allow resources that are provisioned in the control plane VCN 1216 that is contained in the service tenancy 1219, to be deployed or otherwise used in the data plane VCN 1218 that is contained in the customer tenancy 1221.

In other examples, the customer of the IaaS provider may have databases that live in the customer tenancy 1221. In this example, the control plane VCN 1216 can include the data plane mirror app tier 1240 that can include app subnet(s) 1226. The data plane mirror app tier 1240 can reside in the data plane VCN 1218, but the data plane mirror app tier 1240 may not live in the data plane VCN 1218. That is, the data plane mirror app tier 1240 may have access to the customer tenancy 1221, but the data plane mirror app tier 1240 may not exist in the data plane VCN 1218 or be owned or operated by the customer of the IaaS provider. The data plane mirror app tier 1240 may be configured to make calls to the data plane VCN 1218, but may not be configured to make calls to any entity contained in the control plane VCN 1216. The customer may desire to deploy or otherwise use resources in the data plane VCN 1218 that are provisioned in the control plane VCN 1216, and the data plane mirror app tier 1240 can facilitate the desired deployment, or other usage of resources, of the customer.

In some embodiments, the customer of the IaaS provider can apply filters to the data plane VCN 1218. In this embodiment, the customer can determine what the data plane VCN 1218 can access, and the customer may restrict access to public Internet 1254 from the data plane VCN 1218. The IaaS provider may not be able to apply filters or otherwise control access of the data plane VCN 1218 to any outside networks or databases. Applying filters and controls by the customer onto the data plane VCN 1218, contained in the customer tenancy 1221, can help isolate the data plane VCN 1218 from other customers and from public Internet 1254.

In some embodiments, cloud services 1256 can be called by the service gateway 1236 to access services that may not exist on public Internet 1254, on the control plane VCN 1216, or on the data plane VCN 1218. The connection between cloud services 1256 and the control plane VCN 1216 or the data plane VCN 1218 may not be live or continuous. Cloud services 1256 may exist on a different network owned or operated by the IaaS provider. Cloud services 1256 may be configured to receive calls from the service gateway 1236 and may be configured to not receive calls from public Internet 1254. Some cloud services 1256 may be isolated from other cloud services 1256, and the control plane VCN 1216 may be isolated from cloud services 1256 that may not be in the same region as the control plane VCN 1216. For example, the control plane VCN 1216 may be located in “Region 1,” and cloud service “Deployment 8,” may be located in Region 1 and in “Region 2.” If a call to Deployment 8 is made by the service gateway 1236 contained in the control plane VCN 1216 located in Region 1, the call may be transmitted to Deployment 8 in Region 1. In this example, the control plane VCN 1216, or Deployment 8 in Region 1, may not be communicatively coupled to, or otherwise in communication with, Deployment 8 in Region 2.

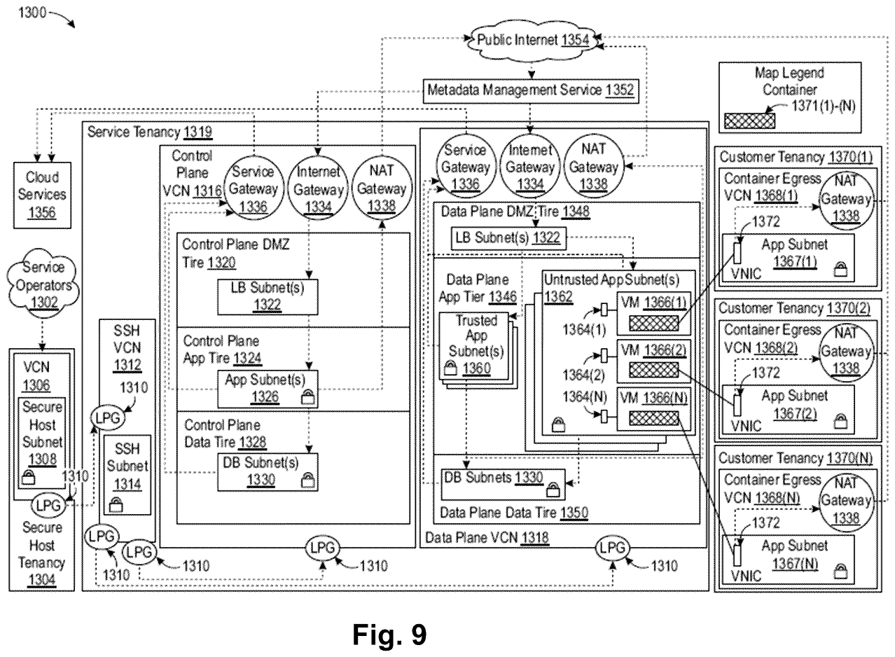

FIG. 9 is a block diagram 1300 illustrating another example pattern of an IaaS architecture, according to at least one embodiment. Service operators 1302 (e.g. service operators 1102) can be communicatively coupled to a secure host tenancy 1304 (e.g., the secure host tenancy 1104) that can include a virtual cloud network (VCN) 1306 (e.g., the VCN 1106) and a secure host subnet 1308 (e.g., the secure host subnet 1108). The VCN 1306 can include an LPG 1310 (e.g., the LPG 1110) that can be communicatively coupled to an SSH VCN 1312 (e.g., the SSH VCN 1112) via an LPG 1310 contained in the SSH VCN 1312. The SSH VCN 1312 can include an SSH subnet 1314 (e.g., the SSH subnet 1114), and the SSH VCN 1312 can be communicatively coupled to a control plane VCN 1316 (e.g., the control plane VCN 1116) via an LPG 1310 contained in the control plane VCN 1316 and to a data plane VCN 1318 (e.g., the data plane 1118) via an LPG 1310 contained in the data plane VCN 1318. The control plane VCN 1316 and the data plane VCN 1318 can be contained in a service tenancy 1319 (e.g., the service tenancy 1119).

The control plane VCN 1316 can include a control plane DMZ tier 1320 (e.g. the control plane DMZ tier 1120) that can include load balancer (“LB”) subnet(s) 1322 (e.g., LB subnet(s) 1122), a control plane app tier 1324 (e.g., the control plane app tier 1124) that can include app subnet(s) 1326 (e.g., similar to app subnet(s) 1126), a control plane data tier 1328 (e.g. the control plane data tier 1128) that can include DB subnet(s) 1330. The LB subnet(s) 1322 contained in the control plane DMZ tier 1320 can be communicatively coupled to the app subnet(s) 1326 contained in the control plane app tier 1324 and to an Internet gateway 1334 (e.g., the Internet gateway 1134) that can be contained in the control plane VCN 1316, and the app subnet(s) 1326 can be communicatively coupled to the DB subnet(s) 1330 contained in the control plane data tier 1328 and to a service gateway 1336 (e.g., the service gateway) and a network address translation (NAT) gateway 1338 (e.g., the NAT gateway 1138). The control plane VCN 1316 can include the service gateway 1336 and the NAT gateway 1338.

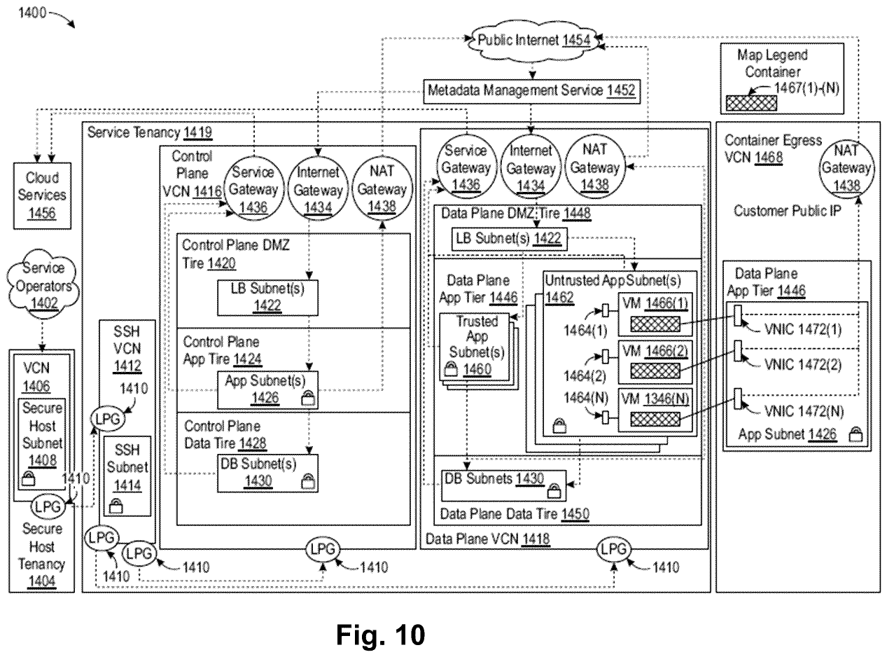

The data plane VCN 1318 can include a data plane app tier 1346 (e.g. the data plane app tier 1146), a data plane DMZ tier 1348 (e.g., the data plane DMZ tier 1148), and a data plane data tier 1350 (e.g., the data plane data tier 1150 of FIG. 10). The data plane DMZ tier 1348 can include LB subnet(s) 1322 that can be communicatively coupled to trusted app subnet(s) 1360 and untrusted app subnet(s) 1362 of the data plane app tier 1346 and the Internet gateway 1334 contained in the data plane VCN 1318. The trusted app subnet(s) 1360 can be communicatively coupled to the service gateway 1336 contained in the data plane VCN 1318, the NAT gateway 1338 contained in the data plane VCN 1318, and DB subnet(s) 1330 contained in the data plane data tier 1350. The untrusted app subnet(s) 1362 can be communicatively coupled to the service gateway 1336 contained in the data plane VCN 1318 and DB subnet(s) 1330 contained in the data plane data tier 1350. The data plane data tier 1350 can include DB subnet(s) 1330 that can be communicatively coupled to the service gateway 1336 contained in the data plane VCN 1318.

The untrusted app subnet(s) 1362 can include one or more primary VNICs 1364(1)-(N) that can be communicatively coupled to tenant virtual machines (VMs) 1366(1)-(N). Each tenant VM 1366(1)-(N) can be communicatively coupled to a respective app subnet 1367(1)-(N) that can be contained in respective container egress VCNs 1368(1)-(N) that can be contained in respective customer tenancies 1370(1)-(N). Respective secondary VNICs 1372(1)-(N) can facilitate communication between the untrusted app subnet(s) 1362 contained in the data plane VCN 1318 and the app subnet contained in the container egress VCNs 1368(1)-(N). Each container egress VCNs 1368(1)-(N) can include a NAT gateway 1338 that can be communicatively coupled to public Internet 1354 (e.g. public Internet 1154).

The Internet gateway 1334 contained in the control plane VCN 1316 and contained in the data plane VCN 1318 can be communicatively coupled to a metadata management service 1352 (e.g. the metadata management system 1152) that can be communicatively coupled to public Internet 1354. Public Internet 1354 can be communicatively coupled to the NAT gateway 1338 contained in the control plane VCN 1316 and contained in the data plane VCN 1318. The service gateway 1336 contained in the control plane VCN 1316 and contained in the data plane VCN 1318 can be communicatively couple to cloud services 1356.

In some embodiments, the data plane VCN 1318 can be integrated with customer tenancies 1370. This integration can be useful or desirable for customers of the IaaS provider in some cases such as a case that may desire support when executing code. The customer may provide code to run that may be destructive, may communicate with other customer resources, or may otherwise cause undesirable effects. In response to this, the IaaS provider may determine whether to run code given to the IaaS provider by the customer.

In some examples, the customer of the IaaS provider may grant temporary network access to the IaaS provider and request a function to be attached to the data plane tier app 1346. Code to run the function may be executed in the VMs 1366(1)-(N), and the code may not be configured to run anywhere else on the data plane VCN 1318. Each VM 1366(1)-(N) may be connected to one customer tenancy 1370. Respective containers 1371(1)-(N) contained in the VMs 1366(1)-(N) may be configured to run the code. In this case, there can be a dual isolation (e.g., the containers 1371(1)-(N) running code, where the containers 1371(1)-(N) may be contained in at least the VM 1366(1)-(N) that are contained in the untrusted app subnet(s) 1362), which may help prevent incorrect or otherwise undesirable code from damaging the network of the IaaS provider or from damaging a network of a different customer. The containers 1371(1)-(N) may be communicatively coupled to the customer tenancy 1370 and may be configured to transmit or receive data from the customer tenancy 1370. The containers 1371(1)-(N) may not be configured to transmit or receive data from any other entity in the data plane VCN 1318. Upon completion of running the code, the IaaS provider may kill or otherwise dispose of the containers 1371(1)-(N).

In some embodiments, the trusted app subnet(s) 1360 may run code that may be owned or operated by the IaaS provider. In this embodiment, the trusted app subnet(s) 1360 may be communicatively coupled to the DB subnet(s) 1330 and be configured to execute CRUD operations in the DB subnet(s) 1330. The untrusted app subnet(s) 1362 may be communicatively coupled to the DB subnet(s) 1330, but in this embodiment, the untrusted app subnet(s) may be configured to execute read operations in the DB subnet(s) 1330. The containers 1371(1)-(N) that can be contained in the VM 1366(1)-(N) of each customer and that may run code from the customer may not be communicatively coupled with the DB subnet(s) 1330.

In other embodiments, the control plane VCN 1316 and the data plane VCN 1318 may not be directly communicatively coupled. In this embodiment, there may be no direct communication between the control plane VCN 1316 and the data plane VCN 1318. However, communication can occur indirectly through at least one method. An LPG 1310 may be established by the IaaS provider that can facilitate communication between the control plane VCN 1316 and the data plane VCN 1318. In another example, the control plane VCN 1316 or the data plane VCN 1318 can make a call to cloud services 1356 via the service gateway 1336. For example, a call to cloud services 1356 from the control plane VCN 1316 can include a request for a service that can communicate with the data plane VCN 1318.

FIG. 10 is a block diagram 1400 illustrating another example pattern of an IaaS architecture, according to at least one embodiment. Service operators 1402 (e.g., service operators 1102) can be communicatively coupled to a secure host tenancy 1404 (e.g., the secure host tenancy 1104) that can include a virtual cloud network (“VCN”) 1406 (e.g., the VCN 1106) and a secure host subnet 1408 (e.g. the secure host subnet 1108). The VCN 1406 can include an LPG 1410 (e.g., the LPG 1110) that can be communicatively coupled to an SSH VCN 1412 (e.g., the SSH VCN 1112) via an LPG 1410 contained in the SSH VCN 1412. The SSH VCN 1412 can include an SSH subnet 1414 (e.g. the SSH subnet 1114), and the SSH VCN 1412 can be communicatively coupled to a control plane VCN 1416 (e.g., the control plane VCN 1116) via an LPG 1410 contained in the control plane VCN 1416 and to a data plane VCN 1418 (e.g., the data plane 1118) via an LPG 1410 contained in the data plane VCN 1418. The control plane VCN 1416 and the data plane VCN 1418 can be contained in a service tenancy 1419 (e.g., the service tenancy 1119).

The control plane VCN 1416 can include a control plane DMZ tier 1420 (e.g., the control plane DMZ tier 1120) that can include LB subnet(s) 1422 (e.g. LB subnet(s) 1122), a control plane app tier 1424 (e.g., the control plane app tier 1124) that can include app subnet(s) 1426 (e.g. app subnet(s) 1126), a control plane data tier 1428 (e.g. the control plane data tier 1128) that can include DB subnet(s) 1430 (e.g., DB subnet(s) 1330). The LB subnet(s) 1422 contained in the control plane DMZ tier 1420 can be communicatively coupled to the app subnet(s) 1426 contained in the control plane app tier 1424 and to an Internet gateway 1434 (e.g. the Internet gateway 1134) that can be contained in the control plane VCN 1416, and the app subnet(s) 1426 can be communicatively coupled to the DB subnet(s) 1430 contained in the control plane data tier 1428 and to a service gateway 1436 (e.g. service gateway 1136) and a network address translation (NAT) gateway 1438 (e.g. NAT gateway 1138). The control plane VCN 1416 can include the service gateway 1436 and the NAT gateway 1438.

The data plane VCN 1418 can include a data plane app tier 1446 (e.g. the data plane app tier 1146), a data plane DMZ tier 1448 (e.g. the data plane DMZ tier 1148), and a data plane data tier 1450 (e.g. the data plane data tier 1150). The data plane DMZ tier 1448 can include LB subnet(s) 1422 that can be communicatively coupled to trusted app subnet(s) 1460 (e.g. trusted app subnet(s) 1360) and untrusted app subnet(s) 1462 (e.g. untrusted app subnet(s) 1362) of the data plane app tier 1446 and the Internet gateway 1434 contained in the data plane VCN 1418. The trusted app subnet(s) 1460 can be communicatively coupled to the service gateway 1436 contained in the data plane VCN 1418, the NAT gateway 1438 contained in the data plane VCN 1418, and DB subnet(s) 1430 contained in the data plane data tier 1450. The untrusted app subnet(s) 1462 can be communicatively coupled to the service gateway 1436 contained in the data plane VCN 1418 and DB subnet(s) 1430 contained in the data plane data tier 1450. The data plane data tier 1450 can include DB subnet(s) 1430 that can be communicatively coupled to the service gateway 1436 contained in the data plane VCN 1418.

The untrusted app subnet(s) 1462 can include primary VNICs 1464(1)-(N) that can be communicatively coupled to tenant virtual machines (VMs) 1466(1)-(N) residing within the untrusted app subnet(s) 1462. Each tenant VM 1466(1)-(N) can run code in a respective container 1467(1)-(N), and be communicatively coupled to an app subnet 1426 that can be contained in a data plane app tier 1446 that can be contained in a container egress VCN 1468. Respective secondary VNICs 1472(1)-(N) can facilitate communication between the untrusted app subnet(s) 1462 contained in the data plane VCN 1418 and the app subnet contained in the container egress VCN 1468. The container egress VCN can include a NAT gateway 1438 that can be communicatively coupled to public Internet 1454 (e.g. public Internet 1154).

The Internet gateway 1434 contained in the control plane VCN 1416 and contained in the data plane VCN 1418 can be communicatively coupled to a metadata management service 1452 (e.g. the metadata management system 1152) that can be communicatively coupled to public Internet 1454. Public Internet 1454 can be communicatively coupled to the NAT gateway 1438 contained in the control plane VCN 1416 and contained in the data plane VCN 1418. The service gateway 1436 contained in the control plane VCN 1416 and contained in the data plane VCN 1418 can be communicatively couple to cloud services 1456.

In some examples, the pattern illustrated by the architecture of block diagram 1400 may be considered an exception to the pattern illustrated by the architecture of block diagram 1300 and may be desirable for a customer of the IaaS provider if the IaaS provider cannot directly communicate with the customer (e.g., a disconnected region). The respective containers 1467(1)-(N) that are contained in the VMs 1466(1)-(N) for each customer can be accessed in real-time by the customer. The containers 1467(1)-(N) may be configured to make calls to respective secondary VNICs 1472(1)-(N) contained in app subnet(s) 1426 of the data plane app tier 1446 that can be contained in the container egress VCN 1468. The secondary VNICs 1472(1)-(N) can transmit the calls to the NAT gateway 1438 that may transmit the calls to public Internet 1454. In this example, the containers 1467(1)-(N) that can be accessed in real-time by the customer can be isolated from the control plane VCN 1416 and can be isolated from other entities contained in the data plane VCN 1418. The containers 1467(1)-(N) may also be isolated from resources from other customers.

In other examples, the customer can use the containers 1467(1)-(N) to call cloud services 1456. In this example, the customer may run code in the containers 1467(1)-(N) that requests a service from cloud services 1456. The containers 1467(1)-(N) can transmit this request to the secondary VNICs 1472(1)-(N) that can transmit the request to the NAT gateway that can transmit the request to public Internet 1454. Public Internet 1454 can transmit the request to LB subnet(s) 1422 contained in the control plane VCN 1416 via the Internet gateway 1434. In response to determining the request is valid, the LB subnet(s) can transmit the request to app subnet(s) 1426 that can transmit the request to cloud services 1456 via the service gateway 1436.

It should be appreciated that IaaS architectures 1100, 1200, 1300, 1400 depicted in the figures may have other components than those depicted. Further, the embodiments shown in the figures are only some examples of a cloud infrastructure system that may incorporate certain embodiments. In some other embodiments, the IaaS systems may have more or fewer components than shown in the figures, may combine two or more components, or may have a different configuration or arrangement of components.

As disclosed, embodiments serve as a pivotal tool in the realm of hospital blood management, providing an extensive overview of all blood product requests from various patient locations throughout the facility. By consolidating data from diverse Inventories, embodiments generate a real-time snapshot of blood demand, allowing healthcare professionals to efficiently track requests and respond with urgency, particularly in life-threatening scenarios.

The features, structures, or characteristics of the disclosure described throughout this specification may be combined in any suitable manner in one or more embodiments. For example, the usage of “one embodiment,” “some embodiments,” “certain embodiment,” “certain embodiments,” or other similar language, throughout this

specification refers to the fact that a particular feature, structure, or characteristic described in connection with the embodiment may be included in at least one embodiment of the present disclosure. Thus, appearances of the phrases “one embodiment,” “some embodiments,” “a certain embodiment,” “certain embodiments,” or other similar language, throughout this specification do not necessarily all refer to the same group of embodiments, and the described features, structures, or characteristics may be combined in any suitable manner in one or more embodiments.

One having ordinary skill in the art will readily understand that the embodiments as discussed above may be practiced with steps in a different order, and/or with elements in configurations that are different than those which are disclosed. Therefore, although this disclosure considers the outlined embodiments, it would be apparent to those of skill in the art that certain modifications, variations, and alternative constructions would be apparent, while remaining within the spirit and scope of this disclosure. In order to determine the metes and bounds of the disclosure, therefore, reference should be made to the appended claims.

What is claimed is:

1. A method of operating a blood bank, the method comprising:
receiving a request for a blood product corresponding to a patient;
determining whether the request is a routine request or an emergency request;
determining an availability of patient history for the patient; and
displaying a color coded listing of parameters corresponding to the request, the parameters including a different color for blood product requests when a designated time has elapsed.

2. The method of claim 1, further comprising:
when the request is the emergency request, displaying an emergency alert.

3. The method of claim 1, further comprising:
when the request is the emergency request, dispensing the request per an emergency protocol.

4. The method of claim 1, further comprising:
when a patient history is available, automatically recommending blood products based on the patient history.

5. The method of claim 1, further comprising:
when a patient history is not available, obtaining blood samples and automatically performing a crossmatch for red blood cells.

6. The method of claim 4, further comprising:
automatically locating the recommended blood products in the blood bank or at a second blood bank in a different facility.

7. The method of claim 6, further comprising:
if the recommended blood products cannot be located, automatically generating an alert to obtain the recommended blood products from an external source.

8. The method of claim 1, further comprising:
using a cloud infrastructure for operating the blood bank, the cloud infrastructure comprising a first virtual cloud network (VCN) comprising a local peering gateway (LPG) communicatively coupled to a secure shell (SSH) VCN via the LPG;
wherein the LPG is contained in a control plane VCN and the SSH VCN is communicatively coupled to a data plane VCN.

9. A computer readable medium having instructions stored thereon that, when executed by one or more processors, cause the processors to operate a blood bank, the operating comprising:
receiving a request for a blood product corresponding to a patient;
determining whether the request is a routine request or an emergency request;
determining an availability of patient history for the patient; and
displaying a color coded listing of parameters corresponding to the request, the parameters including a different color for blood product requests when a designated time has elapsed.

10. The computer readable medium of claim 9, the operating further comprising:
when the request is the emergency request, displaying an emergency alert.

11. The computer readable medium of claim 9, the operating further comprising:
when the request is the emergency request, dispensing the request per an emergency protocol.

12. The computer readable medium of claim 9, the operating further comprising:
when a patient history is available, automatically recommending blood products based on the patient history.

13. The computer readable medium of claim 9, the operating further comprising:
when a patient history is not available, obtaining blood samples and performing a crossmatch for red blood cells.

14. The computer readable medium of claim 12, the operating further comprising:
automatically locating the recommended blood products in the blood bank or at a second blood bank in a different facility.

15. The computer readable medium of claim 14, the operating further comprising:
if the recommended blood products cannot be located, automatically generating an alert to obtain the recommended blood products from an external source.

16. The computer readable medium of claim 9, the operating further comprising:
using a cloud infrastructure for operating the blood bank, the cloud infrastructure comprising a first virtual cloud network (VCN) comprising a local peering gateway (LPG) communicatively coupled to a secure shell (SSH) VCN via the LPG;
wherein the LPG is contained in a control plane VCN and the SSH VCN is communicatively coupled to a data plane VCN.

17. A cloud based system that operates a blood bank, the system comprising:
one or more processors configured to:
receive a request for a blood product corresponding to a patient;
determine whether the request is a routine request or an emergency request;
determine an availability of patient history for the patient; and
display a color coded listing of parameters corresponding to the request, the parameters including a different color for blood product requests when a designated time has elapsed.

18. The system of claim 17, wherein when the request is the emergency request, the processors configured to display an emergency alert.

19. The system of claim 17, wherein when the request is the emergency request, the processors configured to dispense the request per an emergency protocol.

20. The system of claim 17, wherein the system is executed on a cloud infrastructure, the cloud infrastructure comprising a first virtual cloud network (VCN) comprising a local peering gateway (LPG) communicatively coupled to a secure shell (SSH) VCN via the LPG;
wherein the LPG is contained in a control plane VCN and the SSH VCN is communicatively coupled to a data plane VCN.
